(19) United States
(12) Patent Application Publication
(10) Pub. No.: US 2026/0317398 A1
(43) Pub. Date: Oct. 15, 2026

(54) EXTRACTION METHODS USING MICROENCAPSULATION

(71) Applicant: OCEAN ORCHARDS PTY LTD, Yandina Creek, Queensland (AU)

(72) Inventors: Anna HENDRA, Yandina Creek, Queensland (AU); Adrian Edward SPIERINGS, Yandina Creek, Queensland (AU)

(21) Appl. No.: 19/145,048

(22) PCT Filed: Dec. 5, 2023
(86) PCT No.: PCT/AU2023/051259

§ 371 Date: Jul. 1, 2025

(30) Foreign Application Priority Data
Dec. 5, 2022 (AU) 2022903703

(51) Int. Cl. C07K 14/795; C07K 1/14; C07K 1/34; C07K 1/36; C12N 1/066; C12N 1/20; C12N 11/04; C12R 1/01

(52) U.S. Cl. CPC C07K 14/795; C07K 1/145; C07K 1/34; C07K 1/36; C12N 1/066; C12N 1/20; C12N 11/04; C12R 2001/01

(57) ABSTRACT

A method for extracting a compound from organic material, the method including the steps of: encapsulating the organic material in one or more polymeric gel microcapsules having a size less than or equal to about 2000 μm; applying a solvent to the organic material before and/or after encapsulation to provide dissolved compound within the microcapsules; and separating dissolved compound from all or part of the organic material via filtration through the polymeric gel encapsulation.

### FIELD OF THE INVENTION

The present invention relates to a method for extracting one or more compounds from organic material, which incorporates microencapsulation, and also to an atomisation method that may be implemented with same.

### BACKGROUND OF THE INVENTION

Any reference herein to known prior art does not, unless the contrary indication appears, constitute an admission that such prior art is commonly known by those skilled in the art to which the invention relates, at the priority date of this application.

There are numerous methods for compound extraction from organic source/raw materials, and such methods can be improved.

For example, when extracting bioactive compounds from organic matter, like phycocyanin and betanin, high purity can be hard to achieve. Simple low-cost methods typically result in a low yield, whilst other more technically complex methods are costly, and often utilise harmful/toxic solvents. Such solvents are difficult to dispose of safely and are detrimental to the environment. The use of such solvents also often necessitates that any leftover source material/biomass be discarded, as toxic solvent residue may remain therein after extraction.

The present invention seeks to improve the efficacy and outcomes of conventional extraction methods.

### SUMMARY OF THE INVENTION

According to one broad aspect, the present invention provides a method for extracting a compound from organic material, the method including the steps of: (a) encapsulating the organic material in one or more polymeric gel microcapsules having a size less than or equal to about 2000 μm; (b) applying a solvent to the organic material before and/or after encapsulation to provide dissolved compound within the microcapsules; and (c) separating dissolved compound from all or part of the organic material via filtration through the polymeric gel encapsulation.

In some forms, in step (a), the microcapsules are formed by way of ionotropic gelation. In some forms, in step (a), the organic material is encapsulated by spraying an aerosol of a pre-mixture comprising a polymeric gelling agent and the organic material into a crosslinking solution. In some forms, the aerosol is produced by: pressurizing the pre-mixture; and atomizing the pre-mixture. In some forms, the aerosol is produced by delivering the pre-mixture to an atomizing means by way of a pressure gradient or difference. In some forms, the aerosol is produced by: pressurizing the pre-mixture in a vessel; providing an opening in the vessel so as to provide a pressure gradient or difference that forces release of the pre-mixture from the opening; and atomizing the pre-mixture at the opening. In some forms, atomizing comprises providing a gas stream at the opening to atomize the mixture. In some forms, the gas stream is compressed air.

In some forms, step (c) includes mechanically pressing the microcapsules to facilitate filtration. In some forms, step (c) includes applying vacuum extraction to the microcapsules to facilitate filtration. In some forms, applying a solvent after encapsulation in step (b), and step (c), are repeated one or more times.

In some forms, the microcapsules have a size less than or equal to about 1500 μm. In some forms, the microcapsules have a size less than or equal to about 1000 μm. In some forms, the microcapsules have a size in the range of about 100 μm to about 500 μm. In some forms, the microcapsules are substantially spherical.

In some forms, the microcapsules have pore sizes in the range of about 1 nm to about 300 nm. In some forms, the microcapsules have pore sizes in the range of about 2 nm to about 100 nm.

In some forms, the solvent is water. In forms, the pre-mixture further comprises water.

In some forms, the pre-mixture comprises polymeric gelling agent and organic material to be encapsulated (dry weight) in amount of about 1 to about 20 wt % when combined. In some forms, the pre-mixture comprises polymer gelling agent and organic material to be encapsulated (dry weight) in an amount of about 3 to about 10 wt % when combined. In some forms, the pre-mixture comprises polymer gelling agent and organic material to be encapsulated (dry weight) in an amount of about 5 to about 10 wt % when combined.

In some forms, the pre-mixture comprises polymeric gelling agent in an amount of about 1 to about 10 wt %. In some forms, the pre-mixture comprises polymeric gelling agent in an amount of about 1 to about 2.5 wt %.

In some forms, the pre-mixture comprises polymeric gelling agent in an amount of about 1.5 to about 2 wt %. In some forms, the pre-mixture comprises organic material to be encapsulated in amount from about 2 to about 20 wt % (dry weight). In some forms, the pre-mixture comprises organic material to be encapsulated in amount from about 2 to about 10 wt % (dry weight). In some forms, the pre-mixture comprises organic material to be encapsulated in amount from about 2 to about 8 wt % (dry weight). In some forms, the pre-mixture comprises organic material to be encapsulated in amount from about 3 to about 7 wt % (dry weight).

In some forms the pre-mixture has a viscosity up to 1500 mPa s. In some forms the pre-mixture has a viscosity in the range of about 20 to about 1500 mPa s. In some forms, the pre-mixture has a viscosity in the range of about 40 to about 800 mPa s.

In some forms, the polymeric gelling agent comprises a biopolymer. In some forms, the polymeric gelling agent comprises an alginate.

In some forms, the polymeric gelling agent comprises sodium alginate. In some forms, the pre-mixture is formed by mixing the organic material in a gelling agent solution.

In some forms, the gelling agent solution is an aqueous sodium alginate solution having a concentration of about 1 to about 10 wt %. In some forms, the gelling agent solution is an aqueous sodium alginate solution having a concentration of about 3 to about 5 wt %.

In some forms, the crosslinking solution is a salt solution. In some forms, the crosslinking solution is divalent salt solution. In some forms, the salt solution is a calcium salt solution. In some forms, the crosslinking solution is an aqueous calcium chloride solution with a concentration in the range of about 1 to about 10 wt %. In some forms, the crosslinking solution is an aqueous calcium chloride solution with a concentration in the range of about 2 to about 5 wt %.

In some forms, the microcapsules comprise shell component in the range of about 10 to about 65 wt % (dry weight). In some forms, the microcapsules comprise shell component in the range of about 20 to about 50 wt % (dry weight). In some forms, the microcapsules comprise shell component in the range of about 25 to about 35 wt % (dry weight).

In some forms, after encapsulation, the method includes the step of treating the organic material to lyse the cell walls thereof. In some forms, treating the organic material to lyse the cells walls thereof includes freeze-thawing the material, freeze-drying the material, sonicating the material, and/or heating the material.

In some forms, the organic material is bacterial, fungal, algal, plant, or animal material. In some forms, the organic material is microorganism material. In some forms, the organic material comprises cyanobacteria. In some forms, the cyanobacteria is spirulina. In some forms, the spirulina is Arthrospira Maxima or Arthrospira Plantesis. In some forms, the compound to be extracted is phycocyanin.

In some forms, the organic material comprises yeast.

According to a further broad aspect the present invention provides a method for atomizing a mixture comprising organic cell material, the method comprising pressurizing the mixture before atomizing the mixture. In some forms, the pressurized mixture is delivered for atomizing by way of a pressure gradient or difference.

In some forms, the method comprises: pressurizing the mixture in a vessel; providing an opening in the vessel so as to provide a pressure gradient or difference that forces release of the mixture from the opening; and atomizing the mixture at the opening. In some forms, atomizing comprises providing a gas stream at the opening to atomize the mixture. In some forms, the mixture is a suspension or solution. In some forms, the mixture is a suspension of the organic cell material in water.

Wherever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

Wherever it is used, the term “a” and “an” are used to refer to one or more than one (i.e. to at least one) of the grammatical object of the article. By way of example, “an extract” means one extract or more than one extract.

Wherever it is used, the term “about,” is understood to refer to a range of numbers that a person of skill in the art would consider equivalent to the recited value in the context of achieving the same function or result.

It will be appreciated that reference to microcapsule ‘size’ in respect of substantially spherical microcapsules relates to the diameter of the microcapsules. For non-spherical microcapsules, size/diameter is to be determined/approximated using the diameter a circle of equal projection area method (EQPC).

### BRIEF DESCRIPTION OF THE DRAWINGS

FIG. 1 shows a schematic representation of material encapsulated within a porous shell material.

### DETAILED DESCRIPTION

Embodiments of the invention provide a method for extracting a compound from organic material, which comprises encapsulating the organic material in one or more polymeric gel microcapsules having a size less than about 2000 μm. A solvent is applied to the organic material before and/or after encapsulation to provide dissolved compound within the microcapsules. The dissolved compound is then separated from all or part of the organic material via filtration through the polymeric gel encapsulation, providing an extract of high purity.

It will be appreciated that the organic material which becomes encapsulated may otherwise be referred to as the core material, whereas the polymeric gel encapsulation may otherwise be referred to as the encapsulant, shell, wall or coating material. It will also be appreciated that the microcapsules may not have strict separation be outer shell and core material and may comprise, for example, core material interspersed within a polymer matrix. It will also be appreciated that the organic material, prior to encapsulation, may in some cases be pretreated and/or form part of a mixture or solution.

The polymeric gel encapsulation is typically configured/selected to have a pore size or porosity that allows effective separation of the compound from the organic material via filtration through the encapsulation. That is, the pore size or porosity is configured/selected to allow solvent to enter the encapsulation to dissolve the target compound, and to allow the dissolved compound/filtrate to exit out through the pores, whilst larger and non-soluble constituents of the organic material are contained/retained within the encapsulation. For example, in some forms, the polymeric gel encapsulation has pore sizes in the range of about 1 nm to about 300 nm, and in some forms, in the range about 2 nm to about 100 nm.

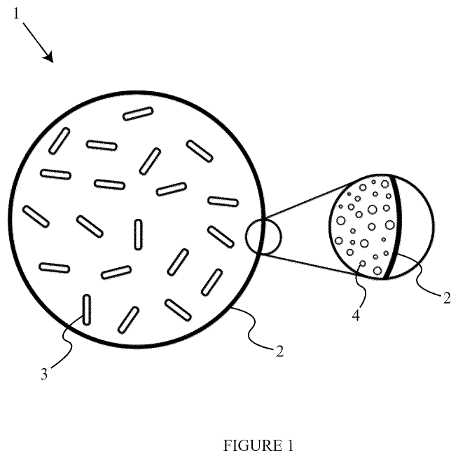

A schematic representation of an encapsulation (1) comprising a core material (3) with pores (4) in encapsulation/shell material (2) is shown in FIG. 1.

By encapsulating the organic material, and by using the encapsulation material as a filter, the inventors have been able to avoid problems normally experienced with conventional filters, like clogging, or the inefficient use of solvent (e.g. water). The extracts obtained are also of a relatively high purity when compared to existing methods, and processing costs are relatively low. The method may also be implemented with non-toxic solvents, like water, which thus facilitates the recycling of left-over raw material (e.g. biomass).

Generally, to facilitate filtration through the encapsulation, a force is applied to the encapsulation/microcapsule to encourage expulsion of the target compound solution/filtrate. In one form, this includes applying pressure to the encapsulation/microcapsule, for example by mechanically pressing the encapsulation to squeeze out or expel filtrate through the pores of the encapsulation/shell material. Those skilled in the art will appreciate that any suitable pressing or squeezing method may be used. In one example, pressing is carried out using a mesh filter, so as to simultaneously separate any expelled filtrate from the encapsulations. Other methods to facilitate filtration may include, for example, applying vacuum extraction (by means of a vacuum pump) to extract or ‘suction out’ the filtrate from the encapsulations, or may use centrifugation.

Whilst the above described facilitation methods are suitable to increase the efficiency/speed of extraction from microcapsules having a size less than or equal to about 2000 μm, such as those utilized in the presently described methods, the inventors anticipate the above described facilitation methods would not be sufficiently effective with larger microcapsule sizes (i.e. >2000 μm), due to the thickness of the capsule walls. Preferably, to provide optimal effectiveness of these facilitation methods, the inventors propose using microcapsules having a size less than or equal to about 1000 μm.

The microcapsules utilized are typically substantially spherical, having diameters less than or equal to about 2000 μm. In some forms, they have a size (i.e. diameter when substantially spherical) less than or equal to about 1500 μm. In some forms, they have a size (i.e diameter when substantially spherical) less than or equal to about 1000 μm. In some forms, they have a size (i.e diameter when substantially spherical) in the range of about 100 to about 500 μm.

Depending on the source/starting organic material, and target compound to be extracted, solvent may be re-applied once the filtrate is substantially flushed/expelled from the encapsulation. This additional solvent dissolves more target compound, and then as before, typically with subsequent pressing etc., more filtrate/target compound may be extracted/expelled. It will be appreciated only a limited amount of solvent is capable of being received into the encapsulation/microcapsule, and so, once saturated, the solvent needs to be replaced to collect/dissolve more target compound. Thus, flushing with fresh solvent may be repeated several times. In such cases wherein the target compound is a pigment, like phycocyanin or betanin, collection and extraction/expulsion of target compound may be repeated until the expelled filtrate is no longer coloured.

In some examples the polymeric gel encapsulation comprises a biopolymer or a mixture of biopolymers. In one example, the encapsulation material comprises an alginate. The alginate may, for example, be calcium or zinc alginate. It will be appreciated that the encapsulation material may vary widely, and other examples of encapsulation material may include but are not limited to: gums, such as gum acacia, agar, or carrageenan; pectins; gelatin; celluloses such as ethyl cellulose; and mixtures of pectin and alginate.

Microencapsulation of the organic material may be achieved by various encapsulation processes. In some forms, encapsulation is achieved by ionotropic gelation. Generally speaking, encapsulation by ionotropic gelation comprises first preparing a pre-mixture comprising the material to be encapsulated with a polymeric gelling agent (typically a polyelectrolyte), and then dispersing the pre-mixture into a solution containing a suitable counter ion such that crosslinking and gel formation occurs. The formed gel encapsulating the organic material to provide microcapsules therearound.

The step of dispersing the pre-mixture in the crosslinking solution may be carried out by various means but in one form, comprises atomizing/aerosolizing the pre-mixture and spraying it into the crosslinking solution. That being said, it is noted that conventional atomization techniques have issues damaging certain organic material, like microorganism or plant cells. For example, mechanical pumping of the pre-mixture to the atomization means/atomization nozzle, and the atomization process itself, often ruptures cell walls prior to encapsulation, resulting in premature release/loss of target compound. When the cell walls are broken prematurely, there is often significant loss of target compound, often into the crosslinking solution, which can be difficult to recover without resorting to expensive chromatography or dialysis.

To address this, the inventors have additionally developed a complementary atomization method wherein the encapsulation pre-mixture is delivered to the atomizing means/atomization nozzle in a way that substantially preserves cell integrity.

The novel method requires first pressurizing the encapsulation pre-mixture, before it is atomized. Pre-pressurization of the pre-mixture allows for the pre-mixture to be delivered to the atomizing means/atomizer by way of a pressure gradient or difference. For example, the encapsulation pre-mixture is typically first pressurized in a vessel. An opening is then provided in the vessel which provides a pressure gradient or difference that forces release of the pre-mixture from the opening (i.e. there is lower pressure at the opening). Atomizing of the pre-mixture by way of an atomizer or atomization means is then carried out at the opening. Typically, atomizing comprises providing a gas stream, like compressed air, at the opening to atomize the pre-mixture. It will be appreciated that generally the pre-mixture is pressurized to a level that provides a suitable pressure gradient/difference to allow for effective delivery to the atomization means at the opening, and in some cases, this may be capped to avoid damage to the organic material (e.g. avoid cell wall lysing). In some examples, the pre-mixture may be pressurized to a pressure in the range of about 15 to about 500 PSI. In some examples, the pre-mixture may be pressurized to a pressure in the range of about 15 to about 200 PSI. In some examples, the pre-mixture may be pressurized to a pressure in the range of about 30 to about 150 PSI. In some examples, the pre-mixture may be pressurized to a pressure in the range of about 40 to about 100 PSI. In some examples, the pre-mixture may be pressurized to a pressure in the range of about 40 to about 60 PSI.

For example, in one implementation where microorganism material might be encapsulated, the encapsulation pre-mixture is placed into a pressure vessel. This vessel once pressurized, also pressurizes the premixture contained within. An opening is created at an atomization point, which is connected to the pressure vessel, and the pressurized mixture is drawn towards the lower pressure at the opening. This method of moving the mixture, as opposed to mechanically pumping, protects the cells of the microorganisms contained within the mixture. Once the pressurized pre-mixture reaches the atomization point at the opening, it is blasted with air to atomize it into tiny, microscopic droplets. It is proposed by the inventors that the pressure within the pre-mixture acts as a counter force to the force of the blasted air, helping to protect the cells of the microorganisms from the forces applied during the atomization process. The preservation of the cells allows them to be later lysed at a determined point (i.e. within the microcapsules), leaving to very little loss of the valuable extracts contained within those cells during the encapsulation process, thus allowing greater yields.

Thus, in some examples, after encapsulation, the method includes the step of treating the organic material to lyse the cell walls thereof within the microcapsules. The target compound thereafter becoming more accessible within the microcapsule. Treating the organic material to lyse the cells walls thereof may include freeze-thawing the material, freeze-drying the material, sonicating the material, and/or heating the material.

The ability to use atomization, as opposed to other methods of dispersion, like using a gravity dropper, allows pre-mixtures to be used which have greater viscosity. This in turn allows allow for larger amounts of the polymeric gelling agent and organic material to be encapsulated to be incorporated in the pre-mixture, such that more microcapsules with complete coverage of the core material are provided, making the process overall more efficient and effective. It also allows the microencapsulation process to be more easily automated and scaled.

In some examples, the pre-mixture comprises polymeric gelling agent and organic material to be encapsulated (dry weight) in amount of about 1 to about 20 wt % when combined. In some examples, the pre-mixture comprises polymer gelling agent and organic material to be encapsulated (dry weight) in an amount of about 3 to about 10 wt % when combined. In some examples, the pre-mixture comprises polymer gelling agent and organic material to be encapsulated (dry weight) in an amount of about 5 to about 10 wt % when combined.

In some examples, the pre-mixture comprises polymeric gelling agent in an amount of about 1 to about 10 wt %. In some examples, the pre-mixture comprises polymeric gelling agent in an amount of about 1 to about 2.5 wt %. In some examples, the pre-mixture comprises polymeric gelling agent in an amount of about 1.5 to about 2 wt %.

In some examples, the pre-mixture comprises organic material in amount from about 2 to about 20 wt % (dry weight). In some examples, the pre-mixture comprises organic material in an amount of about 2 to about 10 wt % (dry weight). In some examples, the pre-mixture comprises organic material in amount from about 2 to about 8 wt % (dry weight). In some examples, the pre-mixture comprises organic material in amount from about 3 to about 7 wt % (dry weight).

The methods as described herein allows for viscosities of encapsulation pre-mixture of up to 1500 mPa s. Typically viscosities of the encapsulation pre-mixture are in the range of 20 to 1500 mPa s, and in some examples, are in the range of about 40-800 mPa s.

As would be appreciated to a skilled person, the polymeric gelling agent typically comprises a polyelectrolyte. In some forms the polymeric gelling agent comprises a biopolymer, and may be, for example, an alginate. In one form the polymeric gelling agent comprises sodium alginate.

It will be appreciated that the encapsulation pre-mixture is typically a suspension or solution formed by mixing the organic material to be encapsulated in a gelling agent solution. It will be appreciated that the constituents and concentrations of the gelling agent and crosslinking solutions utilized in an ionotropic gelation process may vary to form encapsulations with different properties/porosities etc. It will also be appreciated that the organic material to be encapsulated may, in some cases, be part of a mixture or solution, before being mixed with the gelling agent solution to assist with dilution and/or reaching a viscosity/concentration that improves efficiency of the ionotropic gelation process.

In some embodiments, the gelling agent (e.g. polyelectrolyte) solution may comprise an alginate, like sodium or potassium alginate. The alginate solution may have concentration in the range of about 1 wt % to about 10 wt %, and in some embodiments, in the range of about 3 wt % to about 5 wt %. In some embodiments the crosslinking (e.g. counter ion) solution may comprise a salt solution, and may comprise salt with a divalent cation such as, for example, a calcium or zinc salt solution. The salt solution may have a concentration in the range of about 1 to about 10 wt %, and in some embodiments, in the range of about 2 wt % to about 5 wt %. Typically, the gelling agent and crosslinking solution are aqueous solutions.

In one particular example, the organic material to be encapsulated is mixed in an aqueous sodium alginate solution to form the encapsulation pre-mixture, and the pre-mixture then subsequently aerosolized and sprayed in an aqueous calcium chloride solution, such that calcium alginate microcapsule are formed which encapsulate the organic material. The concentration of the sodium alginate solution may be in the range of about 1 wt % to about 10 wt %. In some examples, the sodium alginate concentration is in the range of about 3 wt % to about 5 wt %, and in some examples, is about 4 wt %. The concentration of the calcium chloride solution is typically in the range of about 1 wt % to about 10 wt %. In some examples, the calcium chloride concentration is in the range of about 2 wt % to about 5 wt %, and in some examples, is about 3 wt %. In some examples, the organic material, prior to its initial addition to the sodium alginate solution, is mixed with water to provide an initial mixture with a concentration in the range of about 5 to about 20 wt %, that is then combined with the sodium alginate solution in equal parts.

It will be appreciated to a skilled person that there are many encapsulation or microencapsulation processes that may be utilized to encapsulate the organic source/starting material, such as, for example, matrix polymerization, fluidized bed coating, spray drying etc.

In some examples, the microcapsules formed comprise shell component in the range of about 10 to about 65 wt % (dry weight). In some examples, the microcapsules formed comprise in the range of about 20 to about 50 wt % (dry weight) shell component. In some examples the microcapsules formed comprise in the range of about 25 to about 35 wt % (dry weight) shell component.

Typically, solvent is added after encapsulation, with the microcapsules typically being soaked therein for a period of time (typically 2-6 hours, but may be soaked for up to a day or more) to allow sufficient penetration of the microcapsules and dissolution of the target compound. However, in some examples, solvent may not be required after encapsulation, with solvent present in the encapsulation pre-mixture being sufficient to dissolve the target compound.

Generally, the method is an aqueous extraction process wherein the solvent is water, and the target compound is water soluble. Such embodiments, which avoid the use of non-water solvents that could be toxic, provide that any remaining encapsulation/encapsulated material after the target compound has been extracted will not be contaminated with harmful/toxic solvent residue, and so may be readily used for other purposes. For example, the left-over encapsulation/encapsulated material may be used in or as human or animal food, fertiliser or biochar. It may also be freeze dried and milled for preservation.

Similarly, the filtrate/extract, which in such forms would comprise an aqueous solution, may require less post processing. It will be appreciated that the water may in some examples be, but is not limited to, distilled water, demineralised water, or fresh water. Again, the filtrate may be freeze dried and milled for preservation.

The organic source/starting material may vary and may, for example, be bacterial, fungal, algal, plant, or animal material. The organic material to be encapsulated may for example be microorganism material, such as cyanobacteria or yeast. Target compounds to be extracted in such cases may, for example, be bioactive compounds, proteins, peptides or amino acids.

Although, as described previously, there might be inefficiencies and loss of target compound when cells are lysed prior to encapsulation, in some less preferred forms the source/starting organic material is treated in a manner that lyses the cell walls thereof prior to encapsulation, such that any target compounds might later be more easily dissolvable/accessible. Treating the material to damage the cell walls thereof may, for example, include freeze drying and/or milling the material. More generally, the raw or starting organic material is sometimes in any case milled or processed to produce fine particles thereof in preparation for encapsulation.

As described, in seeking to avoid leakage/loss of target compound prior to encapsulation, treatment to facilitate release of target compound e.g. to lyse the cell walls of organic source material etc. is carried out within the encapsulation. Such treatment may for example include freeze-thawing the material, freeze-drying the material, sonicating the material, and/or heating the material. It will also be appreciated that prior to the encapsulation, the organic material may be pre-mixed with a solvent (e.g. water), which may in some cases be part of the gelling agent solution. In some such cases additional solvent may not be required after encapsulation (this is sometime the case when freeze thawing is used).

In one example implementation, the invention may provide a method for extracting phycocyanin from cyanobacteria. Phycocynain is a blue pigment found exclusively in cyanobacteria and is often used in the food, nutraceutical and pharmaceutical industry. The cyanobacteria may be spirulina such as Arthrospira Maxima or Arthrospira Plantesis. Conventional methods struggle to extract phycocyanin with high purity.

In one example of the described method, spirulina is mixed with water, and that mixture then subsequently mixed in a sodium alginate solution. The resultant encapsulation pre-mixture is then aerosolised/atomised and sprayed in fine beads/droplets (typically 100-500 μm) into a calcium chloride bath. The sodium alginate reacts with the calcium chloride to from gel microcapsules of calcium alginate which trap the spirulina inside. The microcapsules are removed from the bath using a mesh filter and rinsed in clean water. The encapsulated spirulina is treated to lyse the cell walls (e.g. by freeze drying, freeze thawing etc.). The microcapsules are then rehydrated with water, which seeps in though the bead pores to dissolve the phycocyanin. The microcapsules are then mechanically pressed, to encourage expulsion of dissolved phycocyanin, which is leached out as filtrate through the pores of the microencapsulation. The pressing may be carried out using a filter (e.g. 34 μm mesh filter) so as to simultaneously expel and separate filtrate from the beads.

As discussed herein, the encapsulation effectively creates a gel filter where the pore size is large enough to let water and phycocyanin pass through, but that contains/retains larger molecules and compounds that are not water soluble. For phycocyanin, the pore sizes are typically larger than about 10 nm. Rehydration and pressing is repeated until the colour of the filtrate is no longer a pure blue, which indicates the concentration of phycocyanin within the beads is substantially exhausted. Advantageously, being an aqueous extraction, the biomass left over is suitable for human or animal consumption or can be used for fertilizer or biochar. The phycocyanin extracted is also of relatively high purity.

In another example, the organic material to be encapsulated may be derived from beetroot and the compound to be extracted may be betanin. Other example implementations, include extraction of polyphenols from lemon myrtle leaf, and extraction of anthocyanins from Davidson plum. Further example implementations target extracting proteins, peptides and amino acids from yeast.

It will also be appreciated that the extracts produced may be incorporated in a variety of food and beverage products, nutraceutical products, medicaments, pharmaceuticals and over-the-counter formulations.

The extraction methods as described herein allow for high purity extraction of target compounds, due to filtration through the microcapsules, and the ability to implement atomisation for the formation of microcapsules which allows for efficiency and scaling. Furthermore, the specialised atomisation methods allow for atomisation in a way that does not prematurely damage the cell walls of organic cell material.

As previously described, the complementary atomization methods developed by the inventors that may be used with the extraction method allow generally for atomizing a mixture comprising organic cell material, to preserve cell wall integrity. The method comprises pressurizing the mixture before then atomizing the mixture. The pressurized mixture is delivered for atomizing by way of a pressure gradient or difference. Typically, such methods comprise pressurizing the mixture in a vessel, and then providing an opening in the vessel to provide a pressure gradient or difference that forces release of the mixture from the opening (i.e. where there is lower pressure). Atomization then occurs at the opening by way of an atomization means. Such means may comprise providing a gas stream at the opening to atomize the mixture or other means. It will be appreciated that the ‘mixture’ to be atomized is typically a suspension or solution (i.e. organic cell material within a carrier solution/solvent), and not necessarily a mixture of multiple different constituents within a carrier solution/solvent (although this is also possible). For example, the mixture may be a suspension of organic cell material (e.g. microorganism material) in water.

It will be appreciated that generally the mixture is pressurized to a level that provides a suitable pressure gradient/difference to allow for effective delivery to the atomization means at the opening, but not to a level that damages the organic cell material (i.e. to avoid cell wall lysing). In some examples, the mixture may be pressurized to a pressure in the range of about 15 to about 500 PSI. In some examples, the mixture may be pressurized to a pressure in the range of about 15 to about 200 PSI. In some examples, the mixture may be pressurized to a pressure in the range of about 30 to about 150 PSI. In some examples, the mixture may be pressurized to a pressure in the range of about 40 to about 100 PSI. In some examples, the mixture may be pressurized to a pressure in the range of about 40 to about 60 PSI.

### Example 1: Extraction of Phycocyanin From Spirulina

Freeze dried and finely milled spirulina was mixed with water to provide a 5 to 20 wt % mixture. The spirulina mixture was then mixed in equal parts with a 4 wt % sodium alginate solution. If the resulting mixture was too thick, filtered water was added to achieve a desired consistency.

The resulting mixture was then aerosolised and sprayed as fine beads/droplets with an approximate diameter of around 100 to 500 μm into to a 2 to 5 wt % calcium chloride bath. Calcium alginate gel beads form which entrap the spirulina.

The calcium alginate beads with spirulina trapped inside were removed from the bath using a 30 to 50 μm mesh filter, and then rinsed in clean water to remove excess calcium.

The beads were then rehydrated to dissolve phycocyanin therein, and manually pressed, to facilitate extraction/filtering out of the phycocyanin.

Rehydration and pressing was repeated until the colour of the filtrate was no longer pure blue, which typically took five repetitions.

### Example 2: Extraction of Betanin From Beetroot

Freeze dried and finely milled beetroot powder was mixed with water to provide a 5 to 20 wt % mixture. The mixture was then mixed in equal parts with a 4 wt % sodium alginate solution. If the resulting mixture was too thick, filtered water was added to achieve a desired consistency.

The resulting mixture was then aerosolised and sprayed as fine beads/droplets with an approximate diameter of around 100 to 500 μm into to a 2 to 5 wt % calcium chloride bath. Calcium alginate gel beads form which entrap the beetroot powder.

The calcium alginate beads with beetroot powder trapped inside were removed from the bath using a 30 to 50 μm mesh filter, and then rinsed in clean water to remove excess calcium.

The beads were then rehydrated to dissolve betanin therein, and manually pressed, to facilitate extraction/filtering out of the phycocyanin.

Rehydration and pressing was repeated until betanin pigmentation of the filtrate was no longer visible, which typically took 6 repetitions.

### Example 3: Extraction of Phycocyanin From Fresh Spirulina

1 L of fresh spirulina paste, with a solids content of 12 wt % was mixed with 1286 g of a 4 wt % sodium alginate solution. 900 g of water was added to achieve the desired consistency.

The encapsulation mixture contained around 3.77 wt % dry weight equivalent spirulina, 1.61 wt % sodium alginate and 94.62 wt % water. The total solids content was 5.38 wt %.

The mixture was added to a vessel which was then pressurised to 40 PSI.

The mixture was then atomised to 100 to 500 μm droplets which were sprayed into a 3 wt % calcium chloride bath.

The calcium alginate-spirulina microencapsulations were collected using a 50 μm filter and rinsed in fresh water.

The microencapsulations were then stored in a freezer and allowed to completely freeze.

The microcapsules were allowed to thaw in 25° C.

The microcapsules were then manually pressed to extract phycocyanin.

The microcapsules were then rehydrated in fresh water and allowed to sit for 24 hours in 6° C.

The microcapsules were then manually pressed again to extract more phycocyanin.

### Example 4: Extraction of Phycocyanin From Fresh Spirulina

1 L of fresh spirulina paste, with a solids content of 12 wt % was mixed with 750 g of a 4 wt % sodium alginate solution. 500 g of water was added to achieve the desired consistency.

The encapsulation mixture contained around 5.33 wt % dry weight equivalent spirulina, 1.33 wt % sodium alginate and 93.33 wt % water. The total solids content was 6.66 wt %.

The mixture was added to a vessel which was then pressurised to 54 PSI.

The mixture was then atomised to 100 to 500 μm droplets which were sprayed into a 5 wt % calcium chloride bath.

The calcium alginate-spirulina microencapsulations were collected using a 50 μm filter and rinsed in fresh water.

The microencapsulations were then stored in a freezer and allowed to completely freeze.

The microcapsules were allowed to thaw in 25° C.

The microcapsules were then manually pressed to extract phycocyanin.

The microcapsules were then rehydrated in fresh water and allowed to sit for 4 hours in 6° C.

The microcapsules were then manually pressed again to extract more phycocyanin.

### Example 5: Extraction of Phycocyanin From Fresh Spirulina

1 L of fresh spirulina paste, with a solids content of 12 wt % was mixed with 750 g of a 4 wt % sodium alginate solution.

The encapsulation mixture contained around 6.86 wt % dry weight equivalent spirulina, 1.71 wt % sodium alginate and 91.43 wt % water. The total solids content was 8.57 wt %.

The mixture was added to a vessel which was then pressurised to 54 PSI.

The mixture was then atomised to 500 to 1000 μm droplets which were sprayed into a 3 wt % calcium chloride bath.

The calcium alginate-spirulina microencapsulations were collected using a 50 μm filter and rinsed in fresh water.

The microencapsulations were then stored in a freezer and allowed to completely freeze.

The microcapsules were allowed to thaw in 25° C.

The microcapsules were then manually pressed to extract phycocyanin.

The microcapsules were then rehydrated in fresh water and allowed to sit for 4 hours in 6° C.

The microcapsules were then manually pressed again to extract more phycocyanin.

### Example 6: Extraction From Activated Yeast

60 g of bakers yeast (Saccharomyces cerevisiae) was mixed with 440 g of room temperature water and allowed to sit for 30 minutes.

The yeast mixture was then mixed with 643 g of a 4 wt % sodium alginate solution. 450 g of water was added to achieve the desired consistency.

The encapsulation mixture contained around 3.77 wt % yeast, 1.61 wt % sodium alginate and 94.62 wt % water. The total solids content was 5.38 wt %.

The mixture was added to a vessel which was then pressurised to 54 PSI.

The mixture was then atomised to 100 to 500 μm droplets which were sprayed into a 3 wt % calcium chloride bath.

The calcium alginate-yeast microencapsulations were collected using a 50 μm filter and rinsed in fresh water.

The microcapsules were then mixed with 1 L of water.

Using a magnetic stirrer, the mixture was heated to 60° C. and continuously stirred. It was kept there for an hour in order to break the cell walls of the yeast.

Once the mixture was allowed to cool the liquid was collected and the microcapsules were then manually pressed to create a water soluble yeast extract.

The microcapsules were then rehydrated in fresh water and allowed to sit for 4 hours in 6° C.

The microcapsules were then manually pressed again to extract more yeast extract.

### Example 7: Extraction From Deactivated Yeast

100 g of unfortified nutritional yeast (Saccharomyces cerevisiae) was mixed with 900 g of room temperature water.

The yeast mixture was then mixed with 1286 g of a 4 wt % sodium alginate solution. 900 g of water was added to achieve the desired consistency.

The encapsulation mixture contained around 3.14 wt % yeast, 1.61 wt % sodium alginate and 95.25 wt % water. The total solids content was 4.75 wt %.

The mixture was added to a vessel which was then pressurised to 54 PSI.

The mixture was then atomised to 100 to 500 μm droplets which were sprayed into a 3 wt % calcium chloride bath.

The calcium alginate-yeast microencapsulations were collected using a 50 μm filter and rinsed in fresh water.

The microcapsules were then manually pressed to create a water soluble yeast extract.

The microcapsules were then rehydrated in 1 L fresh water and allowed to sit for 2 hours in 6° C.

The microcapsules were then manually pressed again to extract more water soluble yeast extract.

The microcapsules were then rehydrated in 1 L fresh water and allowed to sit for 2 hours in 6° C.

The microcapsules were then manually pressed again to extract more water soluble yeast extract.

### Example 8: Extraction of Polyphenols From Lemon Myrtle

100 g of dried and finely milled lemon myrtle leaf was mixed with 900 g of room temperature water.

The lemon myrtle mixture was then mixed with 1286 g of a 4 wt % sodium alginate solution. 900 g of water was added to achieve the desired consistency.

The encapsulation mixture contained around 3.14 wt % lemon myrtle leaf powder, 1.61 wt % sodium alginate and 95.25 wt % water. The total solids content was 4.75 wt %.

The mixture was added to a vessel which was then pressurised to 54 PSI.

The mixture was then atomised to 100 to 500 μm droplets which were sprayed into a 3 wt % calcium chloride bath.

The calcium alginate-lemon myrtle microencapsulations were collected using a 50 μm filter and rinsed in fresh water.

The microcapsules were then manually pressed to create a water soluble lemon myrtle extract rich in polyphenols.

The microcapsules were then rehydrated in 1 L fresh water and allowed to sit for 2 hours in 6° C.

The microcapsules were then manually pressed again to extract more.

### Example 9: Extraction of Anthocyanins Davidson Plum

60 g of dried and finely milled Davidson plum was mixed with 440 g of room temperature water.

The Davidson plum mixture was then mixed with 643 g of a 4 wt % sodium alginate solution. 450 g of water was added to achieve the desired consistency.

The encapsulation mixture contained around 3.77 wt % Davidson plum powder, 1.61 wt % sodium alginate and 94.62 wt % water. The total solids content was 5.38 wt %.

The mixture was added to a vessel which was then pressurised to 54 PSI.

The mixture was then atomised to 100 to 500 μm droplets which were sprayed into a 3 wt % calcium chloride bath.

The calcium alginate-davidson plum microencapsulations were collected using a 50 μm filter and rinsed in fresh water.

The microencapsulations were then stored in a freezer and allowed to completely freeze.

The microcapsules were allowed to thaw in 25° C.

The microcapsules were then manually pressed to create a water soluble Davidson plum extract rich in anthocyanins.

The microcapsules were then rehydrated in 1 L fresh water and allowed to sit for 20 hours in 6 C.

The microcapsules were then manually pressed again to extract more.

This process was repeated one more time.

1. A method for extracting a compound from organic material, the method including the steps of:
(a) encapsulating the organic material in one or more polymeric gel microcapsules having a size less than or equal to about 2000 μm;
(b) applying a solvent to the organic material before and/or after encapsulation to provide dissolved compound within the microcapsules; and
(c) separating dissolved compound from all or part of the organic material via filtration through the polymeric gel encapsulation.

2. A method as claimed in claim 1, wherein in step (a), the microcapsules are formed by way of ionotropic gelation.

3. A method as claimed in claim 2, wherein in step (a), the organic material is encapsulated by spraying an aerosol of a pre-mixture comprising a polymeric gelling agent and the organic material into a crosslinking solution.

4. A method as claimed in claim 3, wherein the aerosol is produced by:
pressurizing the pre-mixture; and
atomizing the pre-mixture.

5. A method as claimed in claim 4, wherein the aerosol is produced by delivering the pre-mixture to an atomizing means by way of a pressure gradient or difference.

6. A method as claimed in claim 4 or 5, wherein the aerosol is produced by:
pressurizing the pre-mixture in a vessel;
providing an opening in the vessel so as to provide a pressure gradient or difference that forces release of the pre-mixture from the opening; and
atomizing the pre-mixture at the opening.

7. A method as claimed in any one of claims 4 to 6, wherein atomizing comprises providing a gas stream at the opening to atomize the mixture.

8. A method as claimed in claim 7, wherein the gas stream is compressed air.

9. A method as claimed in any one of the preceding claims, wherein step (c) includes mechanically pressing the microcapsules to facilitate filtration.

10. A method as claimed in any one of claims 1 to 8, wherein step (c) includes applying vacuum extraction to the microcapsules to facilitate filtration.

11. A method as claimed in any one of the preceding claims wherein applying a solvent after encapsulation in step (b), and step (c), are repeated one or more times.

12. A method as claimed in any one of the preceding claims, wherein the microcapsules have a size less than or equal to about 1500 μm.

13. A method as claimed in any one of the preceding claims, wherein the microcapsules have a size less than or equal to about 1000 μm.

14. A method as claimed in any one of the preceding claims, wherein the microcapsules have a size in the range of about 100 μm to about 500 μm.

15. A method as claimed in any one of the preceding claims, wherein the microcapsules are substantially spherical.

16. A method as claimed in any one of the preceding claims, wherein the microcapsules have pore sizes in the range of about 1 nm to about 300 nm.

17. A method as claimed in claim 16, wherein the microcapsules have pore sizes in the range of about 2 nm to about 100 nm.

18. A method as claims in any one of the preceding claims, wherein the solvent is water.

19. A method as claimed in claim 3, wherein the pre-mixture further comprises water.

20. A method as claimed in any one of claims 3 to 8 and 19, wherein the pre-mixture comprises polymeric gelling agent and organic material to be encapsulated (dry weight) in amount of about 1 to about 20 wt % when combined.

21. A method as claimed in claim 20, wherein the pre-mixture comprises polymer gelling agent and organic material to be encapsulated (dry weight) in an amount of about 3 to about 10 wt % when combined.

22. A method as claimed in claim 21, wherein the pre-mixture comprises polymer gelling agent and organic material to be encapsulated (dry weight) in an amount of about 5 to about 10 wt % when combined.

23. A method as claimed in any one of claims 3 to 8 and 19 to 22, wherein the pre-mixture comprises polymeric gelling agent in an amount of about 1 to about 10 wt %.

24. A method as claimed in claim 23, wherein the pre-mixture comprises polymeric gelling agent in an amount of about 1 to about 2.5 wt %.

25. A method as claimed in claim 24, wherein the pre-mixture comprises polymeric gelling agent in an amount of about 1.5 to about 2 wt %.

26. A method as claimed in any one of claims 3 to 8 and 19 to 25, wherein the pre-mixture comprises organic material to be encapsulated in amount from about 2 to about 20 wt % (dry weight).

27. A method as claimed in claim 26, wherein the pre-mixture comprises organic material to be encapsulated in amount from about 2 to about 8 wt % (dry weight).

28. A method as claimed in claim 27, wherein the pre-mixture comprises organic material to be encapsulated in amount from about 3 to about 7 wt % (dry weight).

29. A method as claimed in any one of claims 3 to 8 and 19 to 28, wherein the pre-mixture has a viscosity up to 1500 mPa s.

30. A method as claimed in claim 29, wherein the pre-mixture has a viscosity in the range of about 20 to about 1500 mPa s.

31. A method as claimed in claim 29 or 30, wherein the pre-mixture has a viscosity in the range of about 40 to about 800 mPa s.

32. A method as claimed in any one of claims 3 to 8 and 19 to 31, wherein the polymeric gelling agent comprises a biopolymer.

33. A method as claimed in claim 32, wherein the polymeric gelling agent comprises an alginate.

34. A method as claimed in claim 33, wherein the polymeric gelling agent comprises sodium alginate.

35. A method as claimed in claim 3 to 8 and 19 to 34, wherein the pre-mixture is formed by mixing the organic material in a gelling agent solution.

36. A method as claimed in claim 35, wherein the gelling agent solution is an aqueous sodium alginate solution having a concentration of about 1 to about 10 wt %.

37. A method as claimed in claim 36, wherein the gelling agent solution is an aqueous sodium alginate solution having a concentration of about 3 to about 5 wt %.

38. A method as claimed in any one of claims 3 to 8 and 19 to 37, wherein the crosslinking solution is a salt solution.

39. A method as claimed in claim 38, wherein the crosslinking solution is a divalent salt solution.

40. A method as claimed in claim 39, wherein the salt solution is a calcium salt solution.

41. A method as claimed in claim 38, wherein the crosslinking solution is an aqueous calcium chloride solution with a concentration in the range of about 1 to about 10 wt %.

42. A method as claimed in claim 41, wherein the crosslinking solution is an aqueous calcium chloride solution with a concentration in the range of about 2 to about 5 wt %.

43. A method as claimed in any one of the preceding claims, wherein the microcapsules comprise shell component in the range of about 10 to about 65 wt % (dry weight).

44. A method as claimed in any one of the preceding claims, wherein the microcapsules comprise shell component in the range of about 20 to about 50 wt % (dry weight).

45. A method as claimed in any one of the preceding claims, wherein the microcapsules comprise shell component in the range of about 25 to about 35 wt % (dry weight).

46. A method as claimed in any one of the preceding claims, wherein, after encapsulation, the method includes the step of treating the organic material to lyse the cell walls thereof.

47. A method as claimed in claim 43, wherein treating the organic material to lyse the cells walls thereof includes freeze-thawing the material, freeze-drying the material, sonicating the material, and/or heating the material.

48. A method as claimed in any one of the preceding claims, wherein the organic material is bacterial, fungal, algal, plant, or animal material.

49. A method as claimed in any one of the preceding claims, wherein the organic material is microorganism material.

50. A method as claimed in any one of the preceding claims, wherein the organic material comprises cyanobacteria.

51. A method as claimed in claim 50, wherein the cyanobacteria is spirulina.

52. A method as claimed in claim 51, wherein the spirulina is Arthrospira Maxima or Arthrospira Plantesis.

53. A method as claimed in any one of the preceding claims, wherein the compound to be extracted is phycocyanin.

54. A method as claimed in any one of the preceding claims, wherein the organic material comprises yeast.

55. A method for atomizing a mixture comprising organic cell material, the method comprising pressurizing the mixture before atomizing the mixture.

56. A method as claimed in claim 55, wherein the pressurized mixture is delivered for atomizing by way of a pressure gradient or difference.

57. A method as claimed in claim 55 or 56, wherein the method comprises:
pressurizing the mixture in a vessel;
providing an opening in the vessel so as to provide a pressure gradient or difference that forces release of the mixture from the opening; and
atomizing the mixture at the opening.

58. A method as claimed in any one of claims 55 to 57, wherein atomizing comprises providing a gas stream at the opening to atomize the mixture.

59. A method as claimed in any one of claims 55 to 58, wherein the mixture is a suspension or solution.

60. A method as claimed in any one of the claims 55 to 59, wherein the mixture is a suspension of the organic cell material in water.
